(19) United States
(12) Patent Application Publication
(10) Pub. No.: US 2026/0249586 A1
(43) Pub. Date: Aug. 27, 2026

(54) Multi-Layer Kinetic Energy Transfer System.

(71) Applicant: Carmelo Neal Jocson Faller, North Las Vegas, NV (US)

(72) Inventor: Carmelo Neal Jocson Faller, North Las Vegas, NV (US)

(73) Assignee: Veridian Flow IP Holding LLC, North Las Vegas, NV (US)

(21) Appl. No.: 19/660,062

(22) Filed: Apr. 27, 2026

(51) Int. Cl. B32B 5/26; A61L 9/12; B32B 15/04

(52) U.S. Cl. CPC B32B 5/26; A61L 9/127; B32B 15/043; B32B 2250/03; B32B 2307/56; B32B 2307/724; B32B 2457/08

(57) ABSTRACT

A multi-layered smart kinetic system for physiological management and biometric telemetry. The system utilizes a seven-layer architecture featuring a 0.999 fine silver micro-mesh bio-interface and a 19 mm vertical spacer for convective cooling. The system further includes a stimuli-responsive fragrance layer integrated within a moisture-wicking substrate, configured to release a mint-based odorant upon the detection of moisture secretion or kinetic displacement. Integrated Edge-AI via a gold-cased flexible PCB provides real-time data processing, while the entire assembly is configured in an elongated teardrop hexagon geometry for multi-axial force dissipation.

### CROSS-REFERENCE TO RELATED APPLICATIONS

This application claims priority to U.S. Non-Provisional Patent Application, filed Apr. 27, 2027, the entire contents of which are incorporated herein by reference.

### FIELD OF THE INVENTION

The present invention relates generally to smart wearable kinetic management systems and, more particularly, to a multi-layered architectural system utilizing high-value conductive materials and specific geometric spacing for thermal and kinetic regulation.

### BACKGROUND OF THE INVENTION

Wearable kinetic systems require a balance of protection, connectivity, and thermal comfort. Conventional designs often fail to integrate electronic components with kinetic dissipation layers effectively.

### SUMMARY OF THE INVENTION

The architecture includes a 0.999 fine silver micro-mesh interface and a gold-cased flexible PCB separated by a precise 19 mm thermal void. The system further incorporates a stimuli-responsive fragrance substrate within the moisture-wicking architecture, configured to release a mint-based odorant in response to moisture secretion and kinetic displacement.

### BRIEF DESCRIPTION OF THE DRAWINGS

FIG. 1 is an exploded perspective view of the Veridian Flow 7-layer smart kinetic diagram illustrating the layer-to-material alignment and the integrated fragrance-releasing substrate.

FIG. 2 is highlighting the elongated teardrop hexagon geometry and the aromatic diffusion pathways through the 19 mm thermal void.”

### DETAILED DESCRIPTION OF THE INVENTION

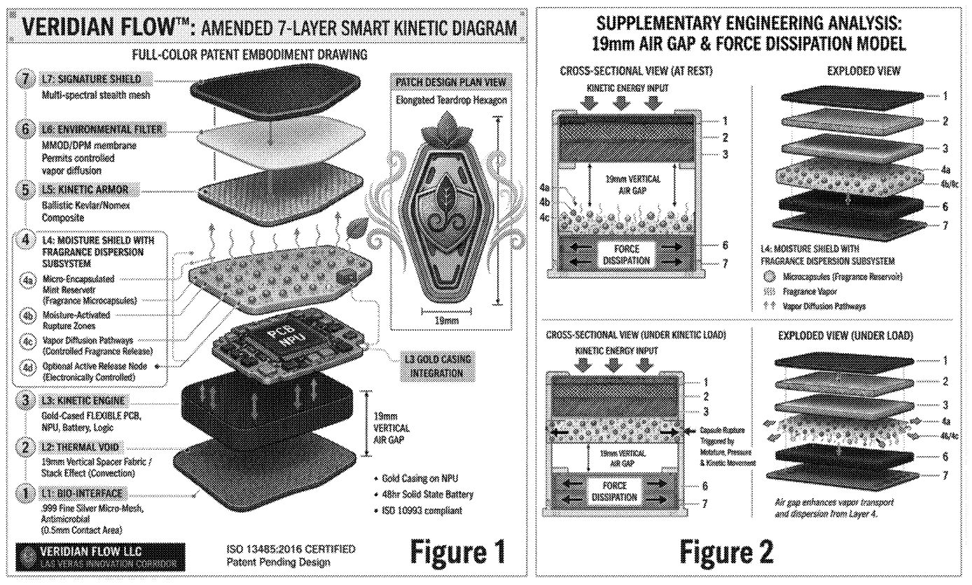

The Veridian Flow system utilizes a 7-layer stack, as seen in FIG. 1. Layer 1 (L1) comprises a 0.999 Fine Silver Micro-Mesh serving as a bio-interface. Layer 2 (L2) is a 19 mm Spacer Fabric defining a Thermal Void for convection management. Layer 3 (L3) consists of a Gold-Cased Flexible PCB. Layer 4 (Moisture Shield) serves as the primary housing for the scent-release mechanism.

The system geometry is defined by an Elongated Teardrop Hexagon as shown in FIG. 2.

Scent Release Mechanism: The fragrance-releasing substrate comprises micro-encapsulated mint particles. These capsules are engineered to rupture or dissolve based on two specific triggers:
Chemical Trigger: Increased pH or moisture from armpit secretions, detected via the silver micro-mesh (Layer 1).
Kinetic Trigger: Friction and heat generated by movement within the 19 mm vertical air gap, as modeled in the force dissipation of FIG. 2.

1. A multi-layer smart kinetic system comprising: a first layer comprising a 0.999 fine silver micro-mesh; a second layer comprising a 19 mm vertical spacer fabric defining a thermal void; and a third layer comprising a gold-cased flexible printed circuit board (PCB).

2. The system of claim 1, wherein the 19 mm vertical spacer fabric is configured to facilitate a stack effect for convection-based thermal management.

3. The system of claim 1, wherein the layers are disposed within a housing having an elongated teardrop hexagon geometry.

4. A multi-layer smart kinetic system comprising: a seven-layer physiological architecture arranged in an elongated teardrop hexagon geometry; a bio-interface layer comprising 0.999 fine silver micro-mesh; a kinetic engine layer comprising a gold-cased flexible printed circuit board (PCB); and a thermal void layer defining a 19 mm vertical air gap disposed between the bio-interface and the kinetic engine. The system of claim 1, wherein the 19 mm vertical air gap is configured to facilitate a convection stack effect for passive thermoregulation. The system of claim 1, wherein the elongated teardrop hexagon geometry is configured to provide multi-axial force dissipation. The system of claim 1, further comprising a stimuli-responsive fragrance-releasing substrate integrated within a moisture-wicking fabric layer, wherein said substrate is configured to release a mint-based odorant upon detection of a threshold level of moisture secretion or a threshold frequency of kinetic displacement.
